(19) United States
(12) Patent Application Publication
(10) Pub. No.: US 2014/0113314 A1
(43) Pub. Date: Apr. 24, 2014

(54) Two Pore Channels as a Therapeutic Target to Protect Against Myocardial Ischemia and as an Adjuvant in Cardiac Surgery

(71) Applicants: Ira S. Cohen, Stony Brook, NY (US); Zhongju Lu, East Setenket, NY (US); Richard B. Robinson, Cresskill, NJ (US); Irvin B. Krukenkamp, South Berwick, ME (US); Peter R. Brink, Setauket, NY (US); Steven J. Feinmark, Haworth, NJ (US)

(72) Inventors: Ira S. Cohen, Stony Brook, NY (US); Zhongju Lu, East Setenket, NY (US); Richard B. Robinson, Cresskill, NJ (US); Irvin B. Krukenkamp, South Berwick, ME (US); Peter R. Brink, Setauket, NY (US); Steven J. Feinmark, Haworth, NJ (US)

(73) Assignees: The Research Foundation of State University of New York, Albany, NY (US); The Trustees of Columbia University in the City of New York, New York City, NY (US)

(21) Appl. No.: 13/689,722

(22) Filed: Nov. 29, 2012

Related U.S. Application Data
(62) Division of application No. 12/296,017, filed on Sep. 18, 2009.
(60) Provisional application No. 60/789,482, filed on Apr. 4, 2006.

(51) Int. Cl. G01N 33/566

(52) U.S. Cl. CPC G01N 33/566
USPC 435/7.21

(57) ABSTRACT

The present invention relates to methods and compositions for modulating the activity of two-pore domain K+ channels (“K2P channels”) as a means for inducing preconditioning protection. Such preconditioning can be used to reduce the effect of ischemia associated with ischemic heart disease, myocardial infarction or cardiac surgery. The invention is based on the discovery that the myoprotective current induced by short periods of ischemia is carried by a non-classical two-pore domain K+ channel.

### CROSS-REFERENCE TO RELATED APPLICATION

This application claims benefit of priority as a divisional application to application Ser. No. 12/296,017, filed Oct. 3, 2008 under 35 U.S.C. §121, which is a national phase (371) application of international application PCT/US2007/008369, filed Apr. 4, 2007 which claims the benefit of priority to provisional applications, 60/789,482 filed Apr. 4, 2006 under 35 U.S.C. §119(e), the entire contents of which are hereby incorporated by reference as if fully set forth herein.

### STATEMENT OF GOVERNMENTAL INTEREST

This invention was made with Government support under Contract No. HL-70161 and HL-28958 awarded by United States Public Health Service—National Heart Lung and Blood Institute. The Government has certain rights in the invention.

### INTRODUCTION

The present invention relates to methods and compositions for modulating the activity of two-pore domain K+ channels (“K2P channels”) as a means for inducing ischemic preconditioning protection. Such preconditioning can be used to reduce the effects of ischemia associated with ischemic heart disease, myocardial infarction or cardiac surgery. The invention is based on the discovery that the myoprotective current induced by short periods of ischemia is carried by a non-classical two-pore domain K+ channel.

### BACKGROUND OF INVENTION

Ischemic heart disease is the leading cause of morbidity and mortality in the Western World and according to the World Health Organization will be the major cause of death in the world by the year 2020 (Murry et al. 1997 Lancet 349: 1498-1504). Ischemic preconditioning (IPC), is defined as one or more short periods of ischemia which can increase the ability of heart to resist subsequent prolonged ischemic injury, and thus has been recognized as a powerful endogenous myoprotective mechanism with significant clinical relevance (Yellon and Downy, 2003, Physiol Rev. 83:1113-1151).

The physiologic basis of IPC has been extensively studied, and there is a general agreement that endogenous triggers (adenosine, bradykinin, opioids, free radicals, et al), mediators (protein kinase C and protein tyrosine kinase) and end-effectors previously thought to be the K_{ATP} channel (the ATP-sensitive K⁺ channel) are all involved in the signaling cascade (Schulz et al., 2001 Cardiorasc. Res. 52:181). Although the triggers and signaling pathways involved in ischemic preconditioning may have been defined, the identity of the surface membrane activated channel has remained unknown. The present invention is based on the discovery that the myoprotective current induced by short periods of ischemia is carried by non-classical two-pore domain K+ channels. Based on this discovery, methods and compositions for modulating the activity of two-pore domain K+ channels (“K2P channels”) are provided as a means for protecting against the effects of ischemia associated with, for example, cardiac disorders, myocardial infarction or cardiac surgery.

### SUMMARY OF THE INVENTION

The present invention relates to methods and compositions for modulating the activity of two-pore domain K+ channels (“K2P channels”) as a means for inducing preconditioning. Such preconditioning can be used to reduce the effects of ischemia associated with ischemic heart disease, myocardial infarction or cardiac surgery. The invention is based on the discovery that the myoprotective current induced by short periods of ischemia is carried by non-classical two-pore domain K+ channels. The invention further relates to screening assays designed to identify compounds that modulate the activity of K2P channels for use in the treatment of ischemic associated disorders.

The invention is based on the discovery that Zn²⁺, a K2P channel blocker, reduces or eliminates the protective current induced by metabolic ischemia or temperature increase. Additionally, it was discovered that the myoprotective mechanism may be associated with reduction in cell size, i.e., shrinking of the cell, in the face of ischemia induced cell swelling.

Accordingly, the present invention relates to methods for inducing preconditioning which serves as a myoprotective mechanism, wherein said method comprises contacting myocytes with a compound capable of modulating the activity of K2P channels. In a preferred embodiment of the invention, the compound is one that is capable of opening K2P channels, thereby permitting an outward current, and effectively protecting myocytes against ischemic damage.

The present invention also provides an in vivo method for protecting myocardium in a mammal from ischemia comprising administrating a compound capable of modulating K2P channel activity in a quantity sufficient to precondition the myocardium against ischemia. Such methods may be used to treat ischemic heart disorders, myocardial infarction or to prevent ischemic injury associated with cardiac surgery.

The invention further provides pharmaceutical compositions comprising a biologically active agent that modulates the activity of a K2P channel in combination with a pharmaceutically acceptable carrier.

The present invention also provides screening assays designed to identify compounds that induce ischemic preconditioning protection based on their ability to modulate the activity of a K2P channel. Modulators of K2P activity can be used to treat subjects suffering from cardiac disorders including, but not limited to, cardiac ischemia and myocardial infarction. Additionally, biologically active agents that modulate the activity of a K2P channel may be utilized during cardiac surgery to prevent ischemic damage normally associated with such surgery.

### BRIEF DESCRIPTION OF THE FIGURES

FIG. 1. Characterization of NaCN induced current from isolated guinea pig ventricular myocytes. A: Sample trace of a large outward current induced by extracellular application of sodium cyanide (NaCN, 2 mM). Note a relatively slow onset phase and sustained phase in the NaCN induced current, and that the current can decay on its own. The cells were held at 0 mV and experiments were performed at 22° C. B: A typical recording of NaCN (2 mM) induced outward current measured in which voltage ramps were applied to obtain the current-voltage relationship Inset of B: The corresponding I/V relationship of NaCN induced current, which is constructed by subtraction of the I/V curve measured at the base (a) from that measured at the peak (b). The cells were held at 0 mV and were subject to hyperpolarizing ramps from +50 mV to −100 mV (250 ms duration) with a frequency of 0.02 Hz.

FIG. 2. Characterization of the K⁺ selective outward current induced by NaCN from isolated guinea pig ventricular myocytes. A: Sample trace of a large outward current induced by extracellular application of sodium cyanide (NaCN, 2 mM). The [K⁺]ₒ was 5.4 mM and the patch pipette [K⁺] was 150 mM. Note a relatively slow onset phase and sustained phase in the NaCN induced current, and that the current can decay on its own. Similar results were observed in all of the cells (n=18) studied in the same conditions. The cells were held at 0 mV and experiments were performed at 22° C. B. The outward current induced by NaCN did not appear when both external and pipette K⁺ were absent. The external K⁺ was absent without substitution and the pipette K⁺ was substituted with L-Aspartic Acid. The pH was adjusted to 7.2 with Trizma Base. Similar results were observed in all of the (n=6) cells studied in the same conditions. C: A typical recording of NaCN induced outward current measured in which voltage ramps were applied to obtain the current-voltage relationship. The [K⁺]ₒ was 3 mM and the pipette [K⁺] was 150 mM. The cells were held at 0 mV and were subject to hyperpolarizing ramps from +50 mV to −100 mV (2s duration) with a frequency of 0.1 Hz (Upper panel). The corresponding I/V relationship of NaCN induced current (Middle Panel), which is constructed by subtraction of the I/V curve measured at the base (a) from that measured at the peak (b) (Lower Panel). D. The I/V curves of the NaCN induced current in three different [K⁺]ₒ were plotted using the same protocol shown in C. E. The linear relationship between averaged reversal potentials and equilibrium potential of K⁺ (E_{K}) calculated with Nernst equation at different [K⁺]ₒ, suggests that the current induced by NaCN is K⁺ selective. Note that the measured reversal potentials are not the same as E_{K} (possibly due to the large intracellular K⁺ loss induced by the NaCN induced-current). The numbers in the parentheses indicate the cells studied.

FIG. 3. Neither sarcolemmal nor mitochondrial _{KATP} channels contribute to NaCN-induced outward current. A: A trace of NaCN induced current. B: Sarcolemmal K_{ATP} channel blocker (glibenclamide, 200 μM) does not prevent the appearance of the NaCN induced current. C: The mitochondrial K_{ATP} channel blocker (5-HD, 200 μM) does not inhibit the NaCN induced current. D: A plot of the average results suggests that neither glibenclamide nor 5-HD displays an inhibitory effect on the NaCN-induced current (unpaired t-test, P>0.05). The currents were normalized to the mean density of NaCN induced current.

FIG. 4. The NaCN induced current is not IK1. A. The NaCN induced current is abolished by a high concentration of Ba2+ (20 mM). B. The NaCN induced current is insensitive to Cs+(3 mm) C. The averaged data indicates that Cs+ has no effect on the NaCN induced current (unpaired t-test, P>0.05). The current densities were normalized to the mean density of the NaCN induced current.

FIG. 5. The NaCN induced current from guinea pig ventricular myocytes is not IK_{ATP} nor IK₁. A: The outward current induced by NaCN can be reversibly abolished by Zn²⁺ (3 mM). Inset of A: Glibenclamide (200 μM) cannot prevent the appearance of the NaCN induced current. B: The IK_{ATP} activated by pinacidil (100 μM) together with low intracellular ATP (0.1 mM) cannot be blocked by Zn²⁺ (3 mM), but is abolished by glibenclamide. C: NO-regulation of the NaCN induced current. Note the NaCN induced current is reduced by the NOS inhibitor, L-NAME (200 μM), and the NaCN induced current is reactivated after washout of Zn²⁺. Inset of C: A typical current trace showing that L-arginine (400 μM) can additionally activate an outward current which is sensitive to Quinidine (1 mM). D: In summary, the average results suggest that the NaCN induced current is not blocked by either the K_{ATP} channel blocker (glibenclamide) or classical K⁺ channel blockers (Ba²⁺ or Cs⁺), it is sensitive to typical K2P channel blockers (both Zn²⁺ and Quinidine) and modulated by NO. In contrast, classical IK_{ATP} is completely blocked by glibenclamide and unaffected by typical K2P channel blockers. All the cells were held at 0 mV.

FIG. 6. Dose-dependent inhibition of NaCN induced current by extracellular application of BaCl₂. A, B and C: Representative recordings of NaCN induced currents were attenuated by extracellular application of Ba²⁺, at a concentration of 5 mM (A), 10 mM (B) and 20 mM (C) respectively. D: Ba²⁺ (20 mM) can prevent the appearance of the NaCN induced current and removal of the Ba²⁺ can unveil an outward current induced by NaCN. E: Dose response relation for Ba²⁺ on the NaCN induced current. The K_{d} =6.1 mM (data was fit to a Langmuir bind K2P channeling isotherm).

FIG. 7. Neither 4-Aminopyridine nor CsCl affect the NaCN induced current. A: Sample trace of NaCN induced current was not reduced by 4-Aminopyridine (4AP, 4 mM). B: Representative recording of NaCN induced current was not affected by extracellular application of CsCl (Cs⁺, 3 mM). C: The averaged date was normalized to the mean density of NaCN induced current. Neither 4AP nor Cs⁺ inhibits the NaCN induced current (unpaired t-test, P>0.05).

FIG. 8. The NaCN induced current from guinea pig ventricular myocytes is sensitive to K2P channel blockers but insensitive to typical K⁺ channel blockers. A: Typical current traces show that neither Ba²⁺ (1 mM, upper panel) nor Cs⁺ (3 mM, lower panel) can prevent the appearance of the NaCN induced current. B: Sample current traces show that both Zn²⁺ (3 mM, upper panel) and quinidine (1 mM, lower panel) can prevent the appearance of the current induced by NaCN.

FIG. 9. The NaCN induced current shares similar biophysical properties with K2P channels. A. Sample trace of NaCN induced current in myocyte when exposing to ramp pulses from −100 mV to =50 mV (250 ms duration) with a frequency of 0.2 Hz. Inset: The I/V relationship is constructed by subtraction if the I/V curve measured at the base prior to activation (a) from that measured at the peak (b). The cells were held at 0 mV. B. Classical I/V curves for outward rectifier, weak inward rectifier (IKATP) and strong inward rectifier (IK₁). C. Typical K2P family members have similar I/V curves to the NaCN induced current.

FIG. 10. Inhibitory effect of ZnCl₂ and quinidine on the NaCN induced current. A: The peak current induced by NaCN can be completely blocked by ZnCl₂ (Zn²⁺, 3 mM) and the inhibition is reversible. B: ZnCl₂ (3 mM) can prevent the appearance of the NaCN induced current. C: The peak current induced by NaCN can be completely abolished by quinidine (0.5 mM). Note a partial reappearance of the NaCN induced current after removal of quinidine. D: Quinidine (0.5 mM) can prevent the appearance of the NaCN induced current and removal of quinidine can initiate a large outward current, which can be blocked by Ba²⁺ (20 mM). E: The peak current amplitudes were normalized to the mean density of the NaCN induced current. Both ZnCl₂ and quinidine can completely inhibit the NaCN induced current (unpaired t-test, P<0.01).

FIG. 11. NaCN induced current is insensitive to methanandamide. A, B and C: Representative recordings of NaCN induced currents were not attenuated by methanandamide, at a concentration of 20 μM (A), 40 μM (B) and 100 μM (C) respectively, but are blocked by Zn²⁺. D: The averaged data suggest that methanandamide (20 μM) does not block the current induced by NaCN (unpaired t-test, P>0.05). Note a significant increase of NaCN induced current by methanandamide at concentrations of 40 and 100 μM (unpaired t-test, P<0.01).

FIG. 12. NaCN induced current was sensitive to external pH. A: A sample trace of NaCN induced outward current in the presence of lower external pH (pHₒᵤₜ=6.0). B: A recording of NaCN induced outward current in the presence of normal external pH (pHₒᵤₜ=7.4). C: Representative trace of an inward shift in holding current induced by NaCN in the presence of higher external pH (pHₒᵤₜ=9.0) followed by a small outward current shift in current. D: The effect of different external pH's on the NaCN induced current is summarized and normalized to pHₒᵤₜ=7.4. The averaged data suggests that lower external pH (pHₒᵤₜ=6.0) significantly increases the amplitudes of the outward current where elevated external pH dramatically reduces the outward current.

FIG. 13. The NaCN induced current is regulated by intracellular pH. A, B, C, D and E: Representative traces of NaCN induced current in the presence of intracellular pH (pHᵢₙ) 5.0 (A), pHᵢₙ 6.0 (B), pHᵢₙ 7.4 (C), pHᵢₙ 9.0 (D) and pHᵢₙ 10.0 (E), respectively. The NaCN induced current in the presence of altered internal pH remains sensitive to quinidine (0.5 mM, B) and Zn²⁺ (5 mM, D). F: Summary of the effects of intracellular pH on the NaCN induced current. The current amplitudes are normalized to the mean density of NaCN induced current in the presence of pHᵢₙ=7.4. Note the increased amplitude of the outward current induced by NaCN with increasing intracellular pH.

FIG. 14. The appearance of the NaCN induced current and the associated shrinkage of myocytes can be prevented by K2P channel blockers. A. Myocyte shrinkage appears concurrently with the NaCN induced current. B and C. Quinidine (0.5 mM) and Zn2+ (3 mM) prevent the appearance of the NaCN induced current and cell shrinkage. The pictures correspond with the numbers shown in the original current traces. Note the appearance of the NaCN induced current and cell shrinkage when quinidine of Zn2+ is removed.

FIG. 15. The characterization of temperature jump (TJ) induced current in guinea pig myocytes. A. Sample trace of TJ induced outward current. Note the current can decay on its own. B. The TJ induced current (upper panel) and its I/V relationships (lower panel). Cells were held at 70 mV and were subject to depolarizing voltage ramps from 100 mV to +50 mV (250 ms duration) with a frequency of 0.1 Hz. Since the current measured at the peak c is out of the current amplitude range of the amplifier, another point near the peak (b) was chosen to construct the I/V curves of the current. C, D, E, and F. Biophysical properties of TREK-1 a heat-activated K2P channel. G. Zn²⁺ can prevent the appearance of TJ induced current. H. The TJ induced current can be partially blocked by quinidine. I. Summary of the inhibitory effects of Zn²⁺ and quinidine on the TJ induced current (unpaired p<0.01, t-test).

### DETAILED DESCRIPTION OF THE INVENTION

Described herein is the discovery that activation of two-pore K+ channels is capable of inducing a myoprotective ischemic preconditioning reaction. The methods and compositions of the invention may be used to reduce the ischemic injury associated with cardiac disorders, myocardial infarction and cardiac surgery. The invention further relates to screening assays designed to identify compounds that modulate the activity of K2P channels and which may be used to induce preconditioning protection. The invention is described in detail in the subsections below.

Modulation of K2P Channels

The present invention encompasses methods for inducing preconditioning through activation of a K2P channel in a mammal Such methods comprise the administration of a biologically active agent capable of modulating the activity of K2P channels to promote preconditioning as may be attributed a means for protecting the myocardium against ischemia associated with heart disorders, myocardial infarction and open heart surgery.

As used herein, “K2P channel” refers to a channel that is characterized as being insensitive to classical K⁺ blockers such as Ba²⁺, Cs⁺ and 4-aminopyridine while being sensitive to Zn²⁺ and quinine derivatives. Additionally, the activity of K2P channels can be modulated by environmental stresses such as heat, protons, oxygen and volatile anesthetics.

As described herein it has been discovered that activation of a K2P channel confers myoprotection against ischemia. Accordingly, the present invention relates to methods for stimulating myoprotection comprising activation of K2P channel protein signal transduction pathways. K2P channels that may be activator for preconditioning include any of the two pore channel family members. In a preferred non-limiting embodiment of the invention the TWIK-2 two pore channel protein can be activated to confer preconditioning protection. Activators of the K2P channel include, but are not limited to NaCN and increases in temperature.

The present invention provides methods and compositions which may be used therapeutically for treatment of various diseases associated with cardiac disorders that result from cardiac ischemia. As used herein, cardiac ischemia refers to a restriction in blood supply to cardiac tissue that results in damage or dysfunction of said tissue. The term “cardiac disorder” as used herein refers to diseases that result from any impairment in the heart's pumping function. This includes, for example, diseases such as angina and myocardial ischemia and infarction characterized by inadequate blood supply to the heart muscle. For further discussion, see Braunwald, Heart Disease: a Textbook of Cardiovascular Medicine, 5th edition, W B Saunders Company, Philadelphia Pa. (1997) (hereinafter Braunwald). The term “cardiomyopathy” refers to any disease or dysfunction of the myocardium (heart muscle) in which the heart is abnormally enlarged, thickened and/or stiffened. As a result, the heart muscle's ability to pump blood is usually weakened. The disease or disorder can be, for example, inflammatory, metabolic, toxic, infiltrative, fibroplastic, hematological, genetic, or unknown in origin. Such cardiomyopathies may result from a lack of oxygen. Other diseases include those that result from myocardial injury which involves damage to the muscle or the myocardium in the wall of the heart as a result of disease or trauma. Myocardial injury can be attributed to many things such as, but not limited to, cardiomyopathy, myocardial infarction, or congenital heart disease. Specific cardiac disorders to be treated also include congestive heart failure, ventricular or atrial septal defect, congenital heart defect or ventricular aneurysm. The cardiac disorder may be pediatric in origin.

The present invention provides for methods for inducing ischemic preconditioning wherein said method comprises contacting cardiomyocytes with an effective amount of a composition comprising a biologically active agent capable of modulating the activity of a K2P channel. Accordingly, the present invention provides a method for treating a subject afflicted with a cardiac disorder resulting from an inadequate blood supply to the heart muscle comprising administering to said subject a composition that modulates K2P channel activity. In preferred embodiments of the invention, the biologically active compound activates the activity of the channel thereby inducing an outward current that serves to protect myocytes against ischemic damage. The composition may be administered to a subject suffering from a cardiac disease in any fashion known to those of skill in the art.

In certain embodiments of the invention the K2P agonist is a lipid, a lipoxygenase metabolite of arachidonic acid or linoleic acid, anisomycin, riluzole, a caffeic acid ester, a tyrphostin, nitrous oxide, propranolol, xenon, cyclopropane, adenosine triphosphate, or copper. In one such embodiment the tyrphostin is tyrphostin 47. In yet another embodiment of the invention K2P agonist that may be used in the practice of the invention include those TREK-1 agonist disclosed in U.S. patent Ser. No. 11/498,343, which is incorporated by reference herein in its entirety.

The compositions of the invention may be administered via an injection into the blood stream, coronary artery, coronary vein, myocardium or pericardial space. Various delivery systems are known and can be used to administer a composition comprising a compound capable of inducing ischemic preconditioning through activation of the K2P channel. Such compositions may be formulated in any conventional manner using one or more physiologically acceptable carriers optionally comprising excipients and auxiliaries. Proper formulation is dependent upon the route of administration chosen.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical sciences” by E. W. Martin. Such compositions will contain a therapeutically effective amount of the therapeutic compound, preferably in purified form, together with a suitable amount of carrier so as to provide the form for proper administration to the patient. The formulation should suit the mode of administration.

The compositions of the invention can be administered by injection into a target site of a subject, preferably via a delivery device, such as a tube, e.g., catheter. In a preferred embodiment, the tube additionally contains a needle, e.g., a syringe, through which the compositions can be introduced into the subject at a desired location.

The compositions may be inserted into a delivery device, e.g., a syringe, in different forms. For example, the compositions of the invention can be suspended in a solution contained in such a delivery device. As used herein, the term “solution” includes a pharmaceutically acceptable carrier or diluent. Pharmaceutically acceptable carriers and diluents include saline, aqueous buffer solutions, solvents and/or dispersion media. The use of such carriers and diluents is well known in the art.

The compositions of the invention may be administered systemically (for example intravenously) or locally (for example directly into the myocardium under echocardiogram guidance, or by direct application under visualization during surgery). For such injections, the compositions may be in an injectable liquid suspension preparation or in a biocompatible medium which is injectable in liquid form and becomes semi-solid at the site of damaged tissue.

In a specific embodiment, it may be desirable to administer the compositions of the invention locally to a specific area of the body; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non porous, or gelatinous material, including membranes, such as silastic membranes, or fibers.

The appropriate concentration of the composition of the invention which will be effective in the treatment of a particular cardiac disorder or condition will depend on the nature of the disorder or condition, and can be determined by one of skill in the art using standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose response curves derived from in vitro or animal model test systems. Additionally, the administration of the compound could be combined with other known efficacious drugs if the in vitro and in vivo studies indicate a synergistic or additive therapeutic effect when administered in combination.

An increase in tissue temperature has also been demonstrated to induce preconditioning protection. Accordingly, when compositions of the invention are administered to the subject in need of treatment, such administration may be carried out in conjunction with a warming of the cardiac tissue.

The progress of the recipient receiving the treatment may be determined using assays that are designed to test cardiac function. Such assays include, but are not limited to ejection fraction and diastolic volume (e.g., echocardiography), PET scan, CT scan, angiography, 6-minute walk test, exercise tolerance and NYHA classification.

Screening Assays

The present invention encompasses screening assays designed to identify modulators of K2P signal transduction pathways for use in preconditioning. Such modulators may be used in the treatment of cardiac disorders based on the ability of K2P activation to induce cardiomyocyte protection.

In accordance with the invention, non-cell based assay systems may be used to identify compounds that interact with, i.e., bind to K2P, and regulate the ischemic activity of cardiomyocytes. Such compounds may be used to regulate cardiomyocyte protection.

Recombinant K2P channel proteins, including peptides corresponding to different functional domains, or K2P channel fusion proteins, may be expressed and used in assays to identify compounds that interact with K2P channels. To this end, soluble K2P channel proteins may be recombinantly expressed and utilized in non-cell based assays to identify compounds that bind to K2P channel proteins. Recombinantly expressed K2P channel proteins, polypeptides or fusion proteins containing one or more of K2P channel protein functional domains may be prepared using methods well known to those of skill in the art, and used in the non-cell based screening assays. For example, a full length K2P channel protein, or a soluble truncated K2P channel protein, e.g., in which the one or more of the cytoplasmic and transmembrane domains is deleted from the molecule, a peptide corresponding to the extracellular domain, or a fusion protein containing the K2P channel proteins extracellular domain fused to a protein or polypeptide that affords advantages in the assay system (e.g., labeling, isolation of the resulting complex, etc.) can be utilized.

The K2P channel protein may also be one which has been fully or partially isolated from cell membranes, or which may be present as part of a crude or semi-purified extract. As a non-limiting example, the K2P channel protein may be present in a preparation of cell membranes. In particular embodiments of the invention, such cell membranes may be prepared using methods known to those of skill in the art.

The principle of the assays used to identify compounds that bind to K2P channel proteins involves preparing a reaction mixture of the K2P channel protein and the test compound under conditions and for time sufficient to allow the two components to interact and bind, thus forming a complex which can be removed and/or detected in the reaction mixture. The identity of the bound test compound is then determined

The screening assays are accomplished by any of a variety of commonly known methods. For example, one method to conduct such an assay involves anchoring the K2P channel protein, polypeptide, peptide, fusion protein or the test substance onto a solid phase and detecting K2P channel protein/test compound complexes anchored on the solid phase at the end of the reaction. In one embodiment of such a method, the K2P channel protein reactant is anchored onto a solid surface, and the test compound, which is not anchored, may be labeled, either directly or indirectly.

In practice, microtitre plates conveniently can be utilized as the solid phase. The anchored component is immobilized by non-covalent or covalent attachments. The surfaces may be prepared in advance and stored. In order to conduct the assay, the non-immobilized component is added to the coated surfaces containing the anchored component. After the reaction is completed, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the solid surface; e.g., using a labeled antibody specific for the previously non-immobilized component.

Alternatively, a reaction is conducted in a liquid phase, the reaction products separated from unreacted components using an immobilized antibody specific for K2P channel proteins, fusion protein or the test compound, and complexes detected using a labeled antibody specific for the other component of the possible complex to detect anchored complexes.

In another embodiment of the invention, computer modeling and searching technologies will permit identification of potential modulators of K2P channel protein signal transduction pathways. The three dimensional geometric structure of the active site may be determined using known methods, including x-ray crystallography, which can determine a complete molecular structure. On the other hand, solid or liquid phase NMR can be used to determine certain intramolecular distances. Any other experimental method of structure determination can be used to obtain the partial or complete geometric structure of the K2P channel protein active site.

Having determined the structure of the K2P channel protein active site, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential K2P channel protein modulating compounds.

In accordance with the invention, non-cell based assays are to be used to screen for compounds that directly activate or inhibit K2P channel protein signal transduction pathway. Such activities include but are not limited to induction or inhibition of ischemic preconditioning. Thus, in a preferred embodiment of the invention, any compounds identified using the non-cell based methods described above, are further tested to determine their ability to modulate ischemic preconditioning.

In accordance with the invention, cell based assay systems can also be used to screen for compounds that modulate the activity of K2P channel protein signal transduction pathways. To this end, cells that endogenously express K2P channel proteins can be used to screen for compounds. Such cells include, for example, cardiomyocytes derived from the heart tissue of a mammal. Alternatively, cell lines, such as HEK293 cells, COS cells, CHO cells, fibroblasts, and the like, genetically engineered to express K2P channel proteins can be used for screening purposes.

In accordance with the invention, a cell-based assay system is provided that can be used to screen for compounds that modulate the activity of K2P channel proteins and, thereby, modulate ischemic preconditioning. The present invention provides methods for identifying compounds that alter one of more of the activities of K2P channel proteins signal transduction pathways, including but not limited to, modulation of ischemic preconditioning. Specifically, compounds may be identified that promote ischemic preconditioning based on their ability to activate K2P channel protein. Alternatively, compounds that inhibit K2P channel protein signal transduction pathways will be inhibitory for ischemic preconditioning.

The present invention provides for methods for identifying a compound that activates the K2P channel protein signal transduction pathway comprising (i) contacting a cell expressing a K2P channel protein with a test compound and measuring the level of K2P channel protein activity; (ii) in a separate experiment, contacting a cell expressing a K2P channel protein with a vehicle control and measuring the level of K2P channel protein activity where the conditions are essentially the same as in part (i), and then (iii) comparing the level of K2P channel protein activity measured in part (i) with the level of K2P channel protein activity in part (ii), wherein an increased level of K2P channel protein activity in the presence of the test compound indicates that the test compound is a K2P channel activator.

In a specific embodiment of the invention, screening assays designed to identify activators of K2P may be utilized to identify compounds that increase K2P activity through increased expression of the channel within a cell membrane. Such compounds may increase expression of K2P channels through increased transcription/translation of K2P genes.

The present invention also provides for methods for identifying a compound that inhibits the K2P channel protein signal transduction pathway comprising (i) contacting a cell expressing a K2P channel protein with a test compound and a known channel activator and measuring the level of K2P channel protein activity; (ii) in a separate experiment, contacting a cell expressing a K2P channel protein with a known channel activator and a vehicle control, where the conditions are essentially the same as in part (i) and then (iii) comparing the level of K2P channel protein activity measured in part (i) with the level of K2P channel protein activity in part (ii), wherein a decrease level of K2P channel protein activity in the presence of the test compound indicates that the test compound is a K2P channel protein inhibitor. K2P channel activators that may be utilized to identify inhibitors include, for example, sodium cyanide (NaCN).

The ability of a test compound to modulate the activity of the K2P channel protein signal transduction pathways may be measured using standard biochemical and physiological techniques. For example, the effect of the test compound on current activity, or function of the cardiomyocytes may be assessed.

In a specific embodiment of the invention, responses normally associated with activation of K+ channel activity may be utilized. Methods of measuring K+ channel activity are well known in the art and most commonly include patch clamp studies which are designed to measure the induced current. Measures of RB efflux and video measurements designed to assess cell shrinkage may be utilized to measure activation of K2P channel activity.

Cell swelling is a prominent feature of ischemic myocardial cell death. Accordingly, cells may be assayed to determine whether changes in cell volume occur in the presence of a test compound. The ability of a test compound to regulate myocyte volume may be measured using methods which include, for example, time-lapse microscopy and parallel patch clamping. Activators of the K2P channel will be those compounds that reduce myocyte swelling when said myocytes are subsequently challenged with an inducer of ischemia.

The assays described above provide a means for identifying compounds which modulate K2P channel signal transduction activity. For example, compounds that affect K2P channel signal transduction activity include but are not limited to compounds that bind to a K2P channel, and either activate the signal transduction activities or block the signal transduction activities. Alternatively, compounds may be identified that do not bind directly to a K2P channel but are capable of altering signal transduction activity by altering the activity of a protein that regulates K2P channel signal transduction activity.

The compounds which may be screened in accordance with the invention include, but are not limited to, small organic or inorganic compounds, peptides, antibodies and fragments thereof, and other organic compounds e.g., peptidomimetics) that bind to a K2P channel and either activate (i.e., agonists) or inhibit the activity of a K2P channel (i.e., antagonists). Compounds that enhance K2P channel signal transduction activities, i.e., agonists, or compounds that inhibit K2P channel signal transduction activities, i.e., antagonists, will be identified. Compounds that bind to proteins and alter/modulate the K2P channel signal transduction activities will be identified.

Compounds may include, but are not limited to, peptides such as, for example, soluble peptides, including but not limited to members of random peptide libraries (see, e.g., Lam, K. S. et al., 1991, Nature 354:82-84; Houghten, R. et al., 1991, Nature 354:84-86); and combinatorial chemistry-derived molecular library made of D- and/or L-configuration amino acids, phosphopeptides (including, but not limited to, members of random or partially degenerate, directed phosphopeptide libraries; (see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope binding fragments thereof), and small organic or inorganic molecules.

Other compounds which may be screened in accordance with the invention include but are not limited to small organic molecules that affect the biological activity, or expression, of the K2P channel genes or some other genes involved in the K2P channel signal transduction pathway (e.g., by interacting with the regulatory region or transcription factors involved in gene expression).

### EXAMPLE

### The Myoprotective Current Induced by Simulated Ischemia is Carried by Two-Pore Domain K+ Channels

The subsection below provides a biophysical and pharmacologic characterization of the myoprotective current induced by metabolic inhibition. The results presented below indicate that application of NaCN induces a current that is carried by a member of the K2P channel family.

Materials and Methods

Single Guinea pig heart cell preparation. Single cardiac myocytes were enzymatically isolated from adult male guinea pig hearts as described in (Gao et al 1992, J Physiol 449:689-704). Guinea pigs, weighing 300 to 500 g, were sacrificed by peritoneal injection with sodium pentobarbital solution (1 ml of 390 mg/ml) in accordance with an approved protocol approved by the IACUC committee at SUNY-Stony Brook. The heart was isolated and placed in Ca²⁺ free Tyrode solution and the aorta was cannulated. The heart was then perfused with 50 ml of Ca2+ free Tyrode solution followed by 100 ml of Tyrode solution containing 30 mol/L CaCl₂ and 0.4 mg/ml collagenase at 37° C. The heart was then placed in Ca²⁺ free Tyrode solution at room temperature for 2 hours. Afterward, a piece of the ventricle was dissected and teased into smaller pieces in Kraft-Brühe (KB) solution (Isenberg et al., 1982 Pflugers Arch 395:6-18) containing (in mM): KCl 83; K2HPO4 30; MgSO4 5; Na-Pyruvic Acid 5; β-OH-Butyric Acid 5; Creatine 5; Taurine 20; Glucose 10; EGTA 0.5; KOH 2; Na₂-ATP 5; pH was adjusted to 7.2 by KOH. The dissociated cells were then kept in KB solution at room temperature for at least 1 hour before the experiment. All solutions were bubbled with 100% O₂. The isolated cells were stored in KB solution. An Axopatch 1D amplifier (Axon Instruments, Inc) and the patch clamp technique were employed to observe cell membrane current. Patch-pipette resistances were 2 to 3 MΩ before sealing. The pipette solution contained (in mM): K-Aspartic Acid 125; KCl 15; KOH 10; MgCl₂ 1; HEPES 10; EGTA 11; Mg-ATP 1; pH was adjusted to 7.2 by KOH. The external Tyrode solution contained (in mM): NaCl 137.7; NaOH 2.3; KCl 4; MgCl₂ 1; HEPES 5; CaCl₂ 1; CdCl₂ 1; Glucose 10; pH was adjusted to 7.4 by NaOH. Under these conditions, the L-type Ca²⁺ current, the Na⁺/Ca²⁺ exchange current and the Na⁺/K⁺ pump current will be absent. In experiments to measure the pH dependence of the currents HEPES (used for pH 6.0 and 10.0) in the bathing medium was replaced with CAPS (pH 10 and 11), Tris (pH 8.5 and 9), or MES (pH 5.0 and 6.0). All experiments were carried out at room temperature (22±1° C.). Sodium cyanide (NaCN) was dissolved in Tyrode solution without glucose and prepared at the target concentration. All patch clamp data were digitized by the data acquisition program pClamp8 (Axon Instruments, Inc) for later analysis. Cell capacitance was obtained for each cell and currents were normalized to cell capacitances.

Chemicals. Methanandamide were purchased from BIOMOL (PA), dissolved in DMSO and then diluted in Tyrode buffer. The final DMSO concentration did not exceed 0.1%. NaCN, glibenclamide, 5-hydroxydecanoic acid (5-HD), collagenase (type II) and other reagents were obtained from Sigma Chemical (St. Louis, Mo.).

Statistical Analysis. All data are presented as mean±SEM. Comparisons between groups were made by unpaired student's T-test. P<0.05 was considered statistically significant. For pooling of pharmacologic data, the peak density of NaCN induced current was measured and normalized to 100%. The effect of agents on the NaCN induced current was then estimated by calculating the ratio of the mean current amplitude in cells studied in the presence of the agent and NaCN to that observed in cells studied in the presence of NaCN alone.

Results

Characterization of the outward current induced by metabolic inhibition. It was previously shown that metabolic inhibition using NaCN can induce a myoprotective effect in the rabbit whole heart model (Inci et al., 2003 Circulation 108 Suppl. 1:II341-11347). Here the effects of NaCN (2 mM) on the biophysical properties of single ventricular myocytes isolated from guinea pig heart were investigated using the whole-cell patch clamp technique. The cells were held at 0 mV and the holding current was monitored.

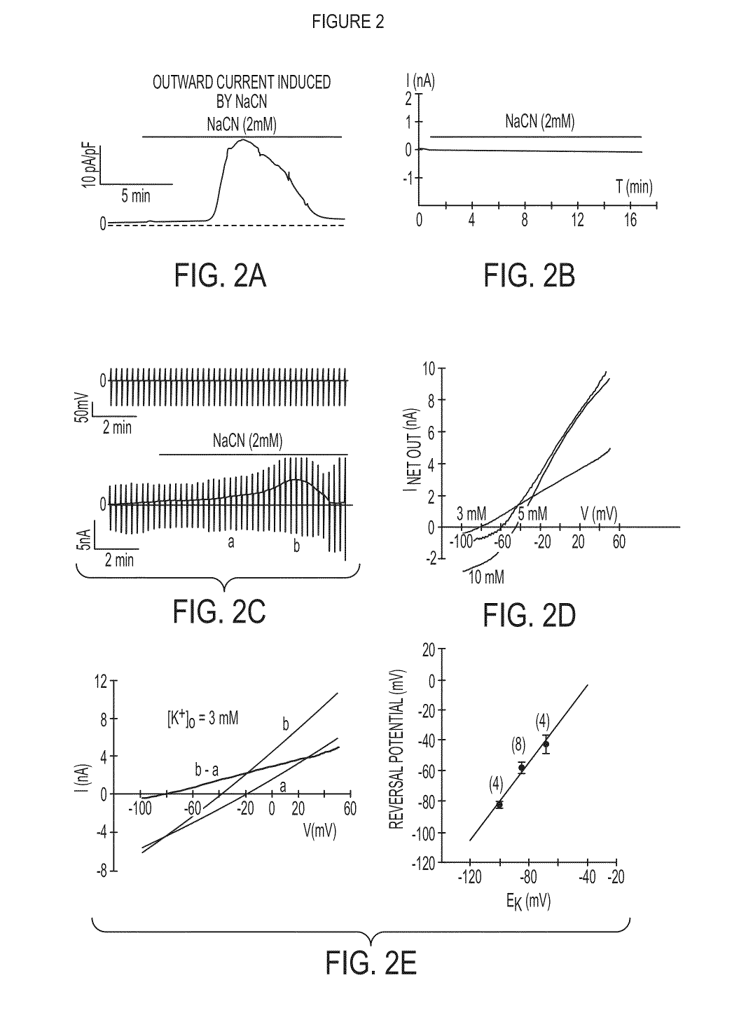

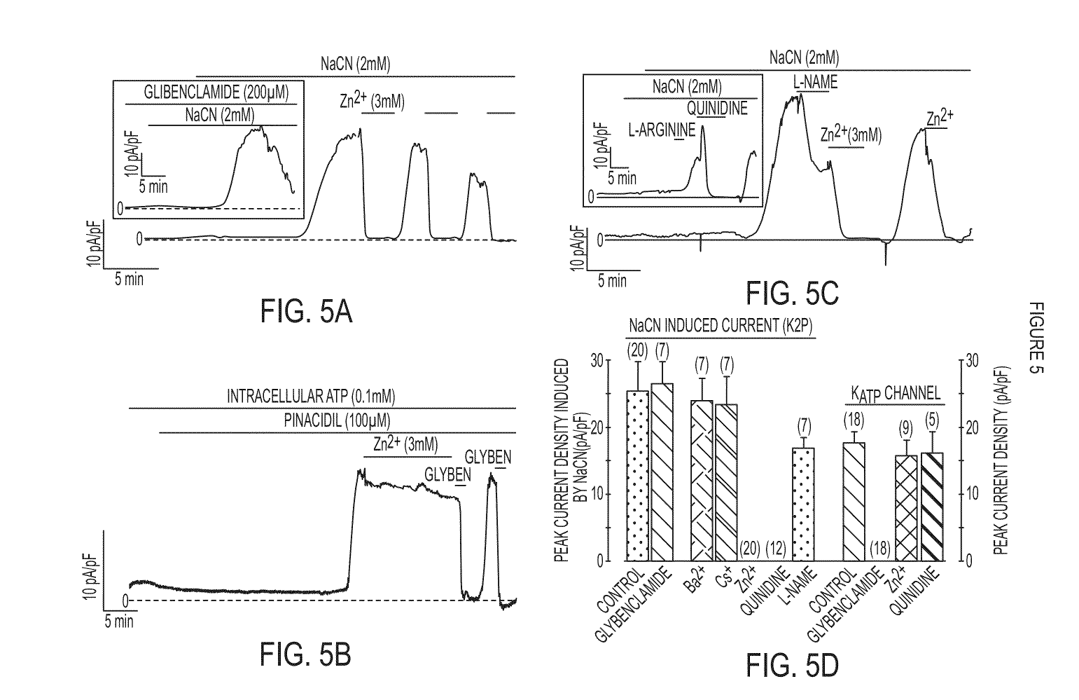

Since the initial report by Murry et al (1986, Murry et al., Circulation 74:1124), ischemic preconditioning has been recognized as a potent endogenous mechanism of myoprotection. In an attempt to investigate the mechanism of this protection, Liu et al. (1997, Am. J. Physiol. 273:H1637) developed a single cell model of metabolic ischemia. Upon exposure to NaCN an outward current is induced in isolated ventricular myocytes. The amplitude of this current increases and the time to appearance of this current shortens when the preparation is first exposed to preconditioning agents (Irie et al., 2003 Circulation 108 Suppl. 1:II341-II347). In the initial study and subsequently (Liu et al., 1997, Am. J. Physiol. 273:H1637), the current activated by metabolic ischemia was identified as IK_{ATP} because it declined when glibenclamide, a blocker of the K_{ATP} channel, was applied at the peak of the response. However, even in the absence of this channel blocker, the current declines on its own (FIG. 2). Further when glibenclamide is included throughout the exposure to NaCN there is no effect on the amplitude of the response (see, below inset of FIG. 5A). This result led to the question whether the channel activated in preconditioning had been correctly identified. In 1996 a new class of ion channels was identified (Fink et al., 1996, EMBO J. 15:6854). These channels were K⁺ specific, also insensitive to classic K⁺ channel blockers like Ba²⁺ and Cs⁺ but instead were blocked by either Zn²⁺ or Quinidine (Kim et al., 2005, Curr. Pharm, Des 11:6854).

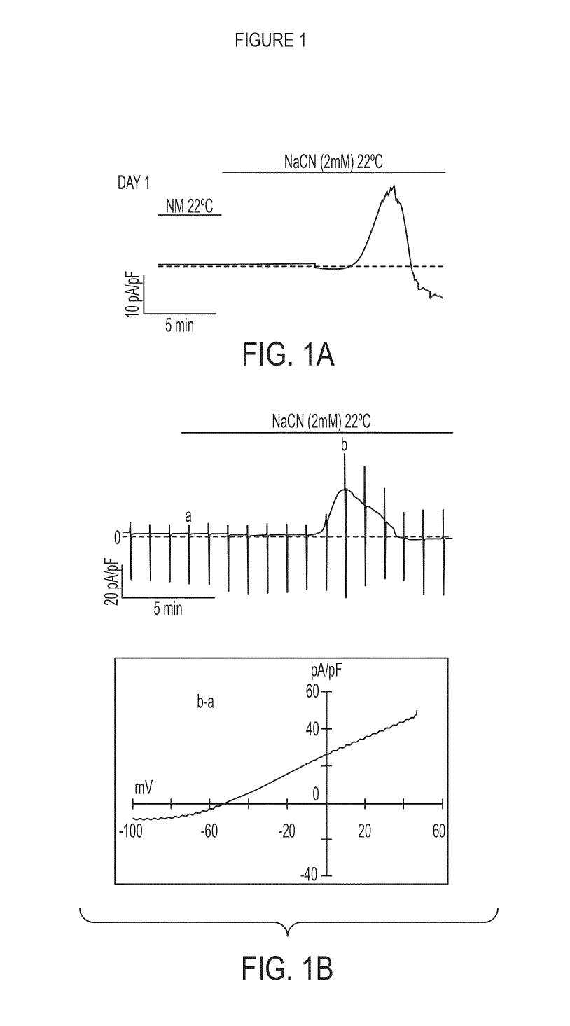

As shown in a representative myocyte (FIG. 1A, application of NaCN induces a large outward current with a slow onset and decay. The peak current which was normalized to the cell capacitance was 19.7±2.0 pA/pF (n=20).

The current/voltage (I/V) relationship of the NaCN induced current was constructed by applying a voltage ramp from +50 mV to −100 mV (250 ms duration) at a frequency of 0.02 Hz. The holding potential between ramps was 0 mV. The upper panel of FIG. 1B shows a sample protocol for a myocyte at 22° C. The corresponding I/V relationship of the NaCN induced current was obtained by subtraction of the I/V curve measured at the baseline from that measured at the peak (The lower panel of FIG. 1B). The I/V relationship of the NaCN induced current is almost linear between −60 mV and +50 mV with a reversal potential around −60 mV. At potentials more negative than −60 mV, there is little increase in the inward current. Similar results were obtained in a total of 12 ventricular myocytes.

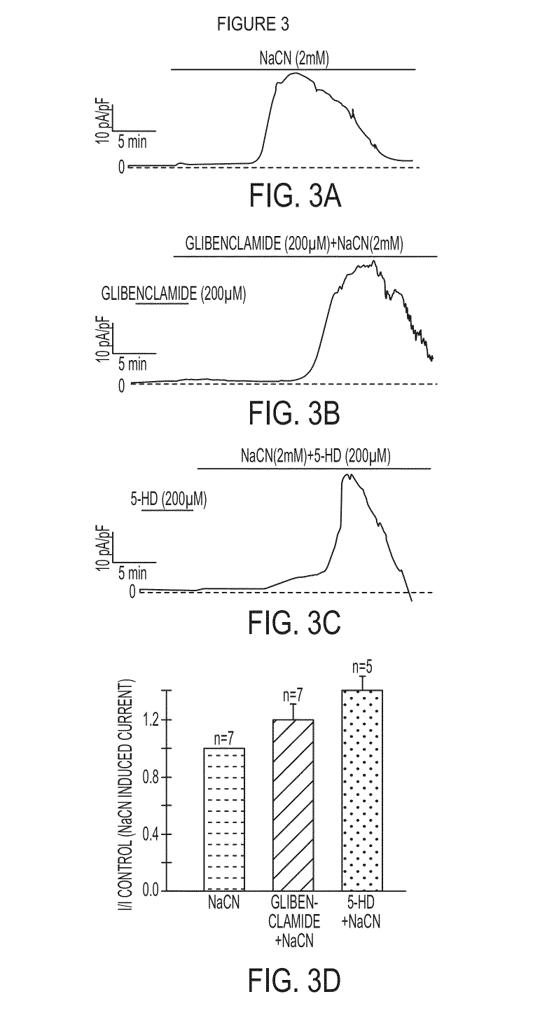

Effect of sarcolemmal- and mitochondrial-K_{ATP} channel blockers on the NaCN induced current. Both sarcolemmal- and mitochondrial-K_{ATP} channel blockers were used to test whether the NaCN induced current was due to the activation of K_{ATP} channels. FIG. 3(A) shows the representative trace of the NaCN induced current in the control condition. As shown in FIG. 3B, NaCN induced current in myocytes could not be abolished by exposure to the sarcolemmal K_{ATP} channel blocker, glibenclamide (200 μM). Similarly, application of the mitochondrial K_{ATP} channel blocker, 5-hydroxydecanoic acid (5-HD) (200 μM) does not result in a reduced current amplitude (FIG. 3C). The peak current densities induced by NaCN are 23.0±2.1 pA/pF (n=7) in the presence of glibenclamide and 24.7±2.6 pA/pF (n=5) when 5-HD was applied. The effects of glibenclamide and 5-HD on normalized peak current density of NaCN induced current for all experiments are plotted on FIG. 3D. The ratio of peak current density induced by NaCN in the presence of glibenclamide or 5-HD to that obtained on exposure to NaCN alone were 1.2±0.1 (n=7), and 1.3±0.1 (n=5), respectively. There are no significant differences induced by exposure to either IK_{ATP} blocker (unpaired T-test, P>0.05).

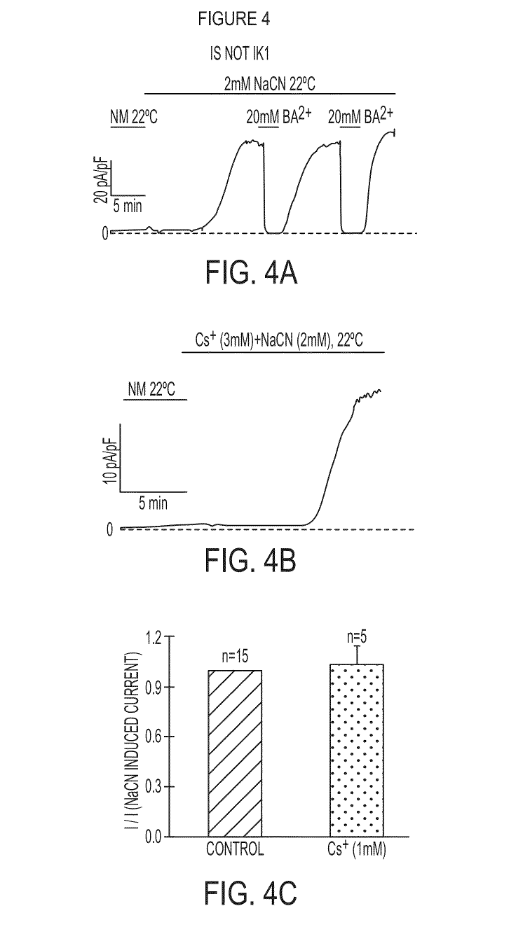

FIG. 4 demonstrates that the NaCN induced current is not IK1. FIG. 4A indicates that the NaCN induced current is abolished by a high concentration of Ba2+ (20 mM). FIG. 4B demonstrates that the NaCN induced current is insensitive to Cs+(3 mm). The averaged data indicates that Cs+ has no effect on the NaCN induced current (unpaired t-test, P>0.05). FIG. 4C. The current densities were normalized to the mean density of the NaCN induced current.

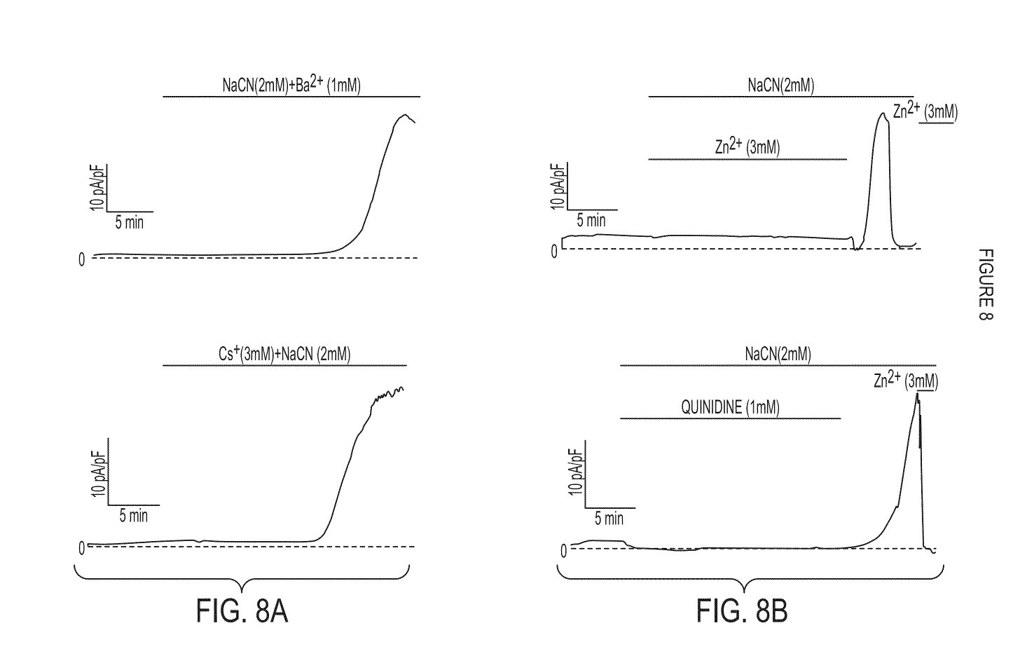

FIG. 5A demonstrates that the current activated by metabolic ischemia is not blocked by glibenclamide but is blocked by Zn²⁺ (also see FIG. 8). Since glibenclamide is a poor blocker of K_{ATP} channels in acidotic conditions we asked whether the current activated by metabolic ischemia might still be K_{ATP} and that this channel might also be blocked by Zn²⁺ or Quinidine. FIGS. 5B and 5D show our results. IK_{ATP} was activated by pinacidil together with a low concentration of intracellular ATP (0.1 mM). This current is identified as IK_{ATP} by its sensitivity to glibenclamide. It is however unaffected by either Zn²⁺ or quinidine. There are at least 15 members in the K2P family, and a number of these channels such as TALK-1 and TALK-2 are directly activated by nitric oxide (5). Given the importance of NO to preconditioning, we examined the effects of an activator (L-Arginine, 400 μM) and an inhibitor (L-NAME, 200 μM) of the NO pathway on the NaCN induced current. The results are provided in FIG. 5C. Activating the NO pathway increases the NaCN induced current while inhibiting the pathway reduces the current. These results (summarized in FIG. 5D) indicate that there is constitutive NO production in guinea pig ventricular myocytes and that the NaCN induced current is a K2P channel that is modulated by NO.

The data indicated that the current initiated by metabolic ischemia is not mediated by the K_{KATP} channel. Instead, an NO sensitive member of the K2P family is implicated. It is well known that the K2P channels help to regulate cell volume (6). It is possible that their role in volume regulation plays a key role in the protection they afford from prolonged ischemia where cell swelling can induce apoptosis (7). With the identification of a novel sarcolemmal channel involved in preconditioning, a new therapeutic target is provided. Other activators of the K2P channels should have the potential to induce preconditioning.

Dose-dependent inhibition of NaCN induced current by Ba²⁺ It was previously demonstrated that the NaCN induced current could be partially blocked by extracellular application of barium (Ba²⁺, 5 mM) (Gao et al. 2005 Biophysical Journal (Abstract) 80:637a). The effect of Ba²⁺ at different concentrations on the NaCN induced current was tested to determine the K_{d} for Ba²⁺ inhibition. In individual experiments, a large outward current was induced by NaCN and different concentrations of Ba²⁺ were then applied in the presence of NaCN. Partial inhibition of the current was obtained by 5-10 mM Ba²⁺ (FIGS. 3A-3B), but increasing the concentration of Ba²⁺ (20-40 mM) led to an almost complete elimination of the NaCN induced current. It was also noticed that the NaCN induced current declined on its own which we observed during the washout of the barium. This effect of Ba²⁺ was both repeatable and reversible (FIG. 3C). Moreover, Ba²⁺ (20 mM) can prevent the appearance of the current when applied before and during exposure to NaCN and the outward current can be induced shortly after removal of Ba²⁺ (FIG. 3D). The percentage of inhibition by Ba²⁺ was plotted as a function of the different concentrations of Ba²⁺ (FIG. 3E). The data were fitted to the Langmuir binding isotherm and yielded a K_{d} of 6.1 mM for Ba²⁺.

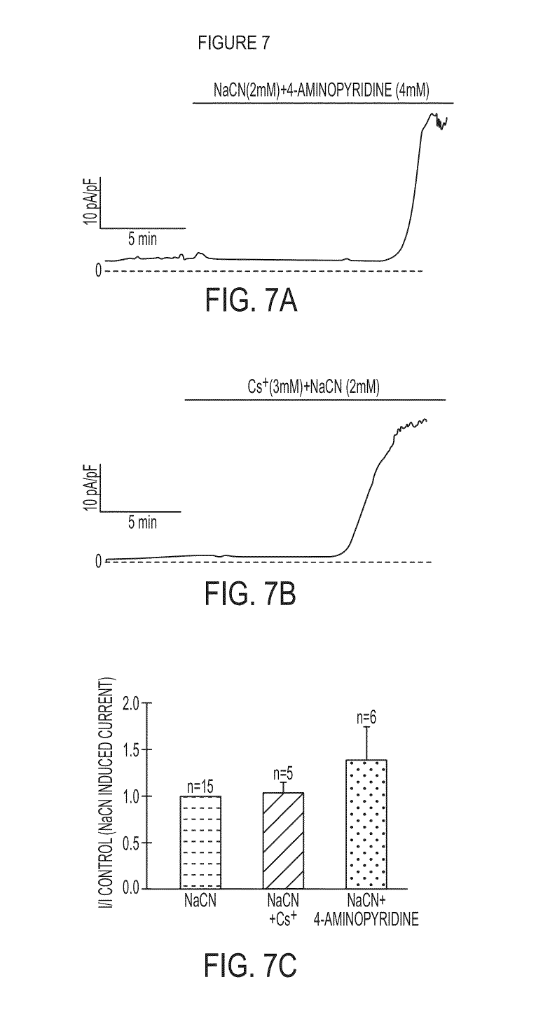

The NaCN induced current was insensitive to classic K⁺ channel blockers. Given that the NaCN induced current is an outward K⁺ current, it was further investigated whether this current was sensitive to other classic K⁺ channel blockers. Neither 4-Aminopyridine (4AP, 4 mM) (FIG. 7A) nor Cs⁺ (3 mM) (FIG. 7B) can prevent the appearance of the NaCN induced current. Furthermore neither blocker reduced the peak current densities which were 19.1±2.4 pA/pF (n=5) with Cs⁺ and 24.1±5.2 pA/pF (n=6) with 4AP, respectively. The peak density of the NaCN induced current in the presence of 4AP and Cs⁺ was normalized and compared to that of NaCN alone (control condition) (FIG. 7C). The averaged data indicates that neither 4AP nor Cs⁺ display a significant inhibitory effect on the NaCN induced current (Unpaired T-test, p>0.05).

FIG. 8 further demonstrates that the NaCN induced current from guinea pig ventricular myocytes is sensitive to K2P channel blockers but insensitive to typical K⁺ channel blockers.

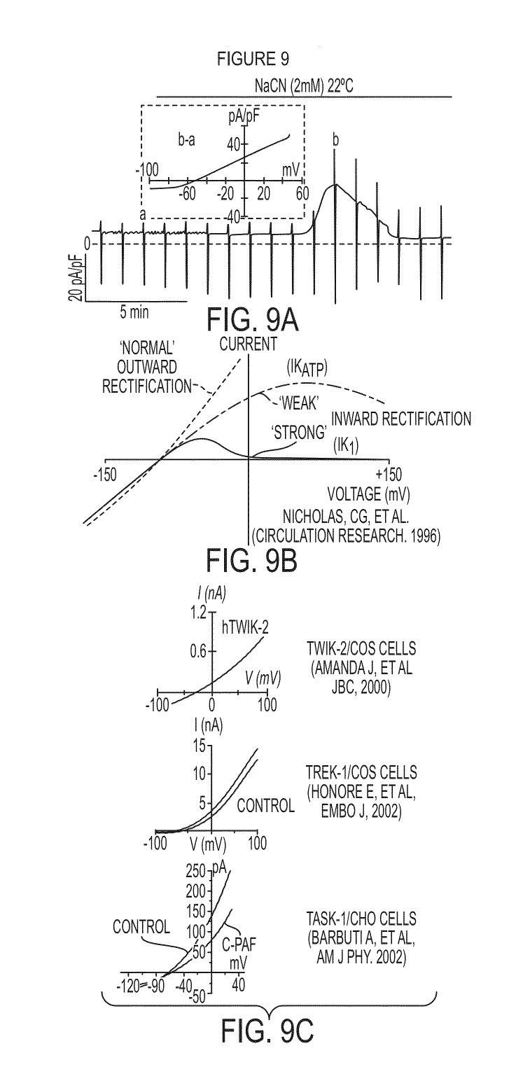

The NaCN induced current shares similar biophysical properties with K2P channels. Sample trace of NaCN induced current in myocyte when exposing to ramp pulses from −100 mV to +50 mV (250 ms duration) with a frequency of 0.2 Hz. Inset: The I/V relationship is constructed by subtraction if the I/V curve measured at the base prior to activation (a) from that measured at the peak (b). The cells were held at 0 mV. FIG. 9A and FIG. 9B demonstrate classical I/V curves for outward rectifier, weak inward rectifier (IKATP) and strong inward rectifier (IK₁). Typical K2P family members have similar I/V curves to the NaCN induced current (FIG. 9C).

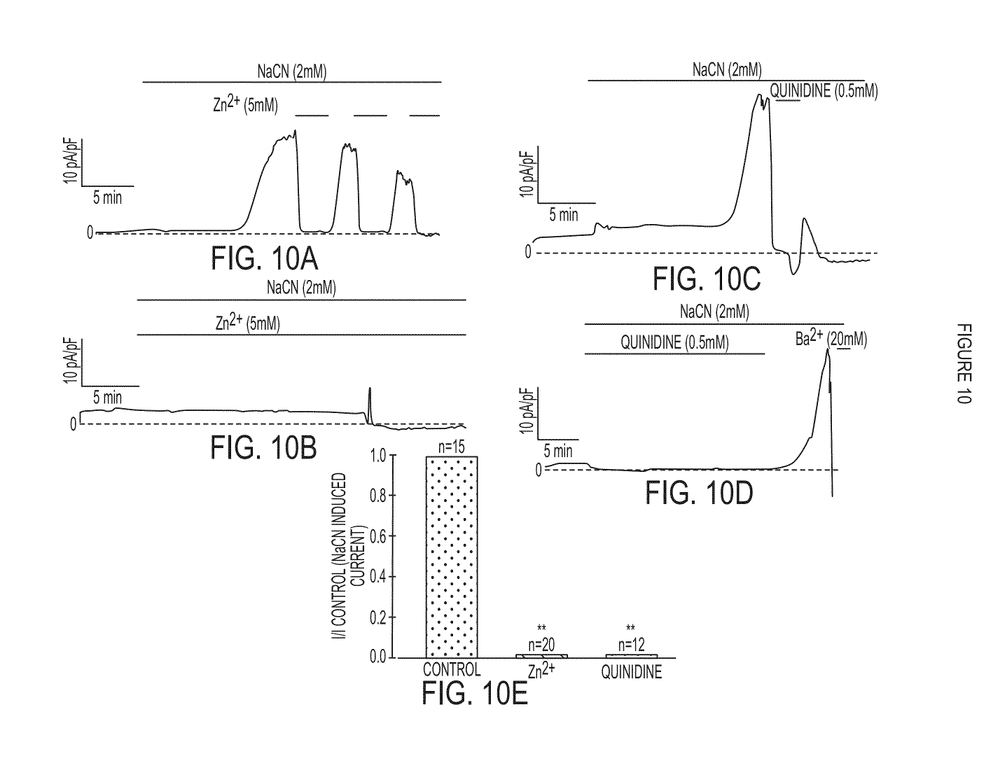

Inhibition of the NaCN induced current by K2P channel blockers. To further identify the NaCN induced current, experiments were conducted to determine whether the current could be modulated by the K2P channel blockers, Zn²⁺ and quinidine (16;21). Zn²⁺ (5 mM) can completely abolish the peak current induced by NaCN, and this inhibitory effect is reversible and reproducible (FIG. 10B). Furthermore, Zn²⁺ can also prevent the appearance of NaCN induced current. Using the same protocols, we found that quinidine (0.5 mM) can both block the peak current induced by NaCN and prevent its occurrence (FIGS. 10C-10D). The normalized data are provided in FIG. 10E. It is clear that the NaCN induced current can be completely abolished by both specific K2P channel blockers.

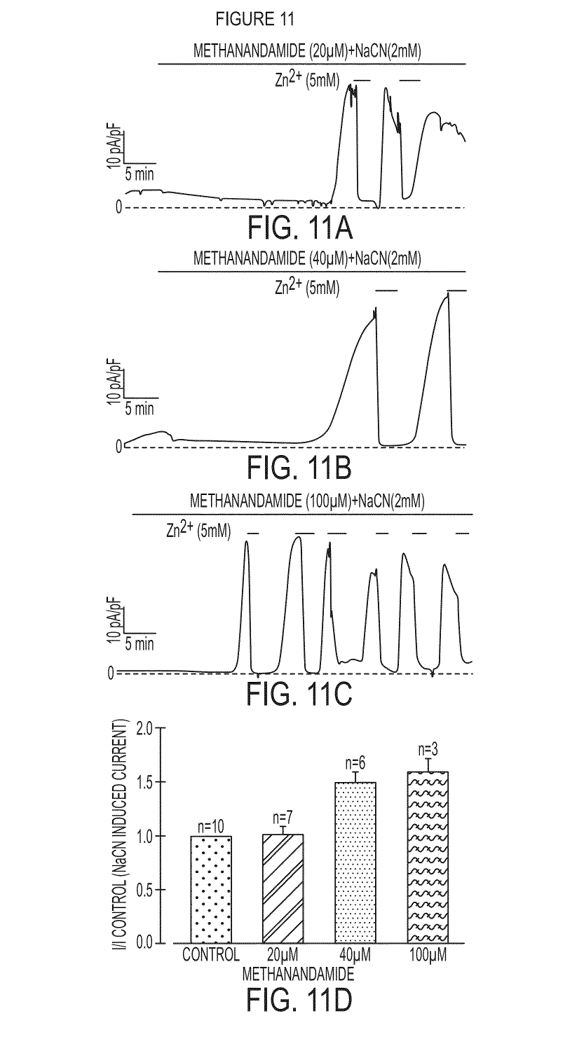

The NaCN induced current is not carried by TASK channels. Although there are blockers for the K2P family, there are relatively few blockers that are selective between family members. One notable exception is methanandamide, a specific blocker of the TASK subfamily (Barbuti et al., 2002 Am J Physiol Heart Cir Physiol 282:H2024-H2030). Experiments were conducted to test whether the NaCN induced current is carried by a TASK family member. Methanandamide at a concentration range from 20 μM to 100 μM does not prevent the appearance of the NaCN induced current. However, the current in these experiments can be abolished by ZnCl₂ (3 mM) (FIG. 11A-11C). The averaged data shows that Methanandamide displays no inhibitory effect on the NaCN induced current. Surprisingly, Methanandamide at higher concentration (40-100 μM) significantly increased rather than decreased the normalized current amplitudes (FIG. 11D) (Unpaired T-test, p<0.01).

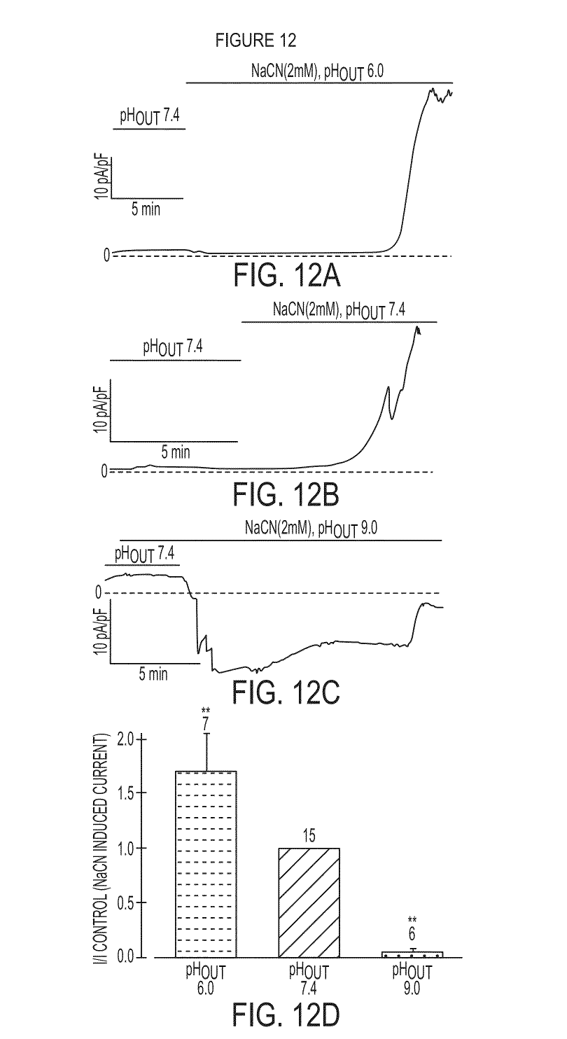

Reducing external pH (pHₒᵤₜ) increases the NaCN induced current. To investigate the influence of external pH on the NaCN induced current, the pH was adjusted to target values prior to experiments. In whole cell mode, the cells were first equilibrated for 5 min in normal Tyrode's solution with a pH of 7.4, and then switched to the target solutions. FIG. 12B shows a representative trace of NaCN induced outward current at an external pH of 7.4 (control condition). When the external pH is reduced from 7.4 to 6.0, NaCN initiates a larger outward current compared to the control condition (FIG. 12A). Exposure to an external pH of 9.0 (FIG. 12C) in the presence of NaCN results in a relatively rapid inward current shift after which a small outward current is activated. In summary, The NaCN induced outward current can be significantly increased by the external acidosis (pHₒᵤₜ 6.0) and inhibited by external alkalosis (pHₒᵤₜ 9.0) (FIG. 12D).

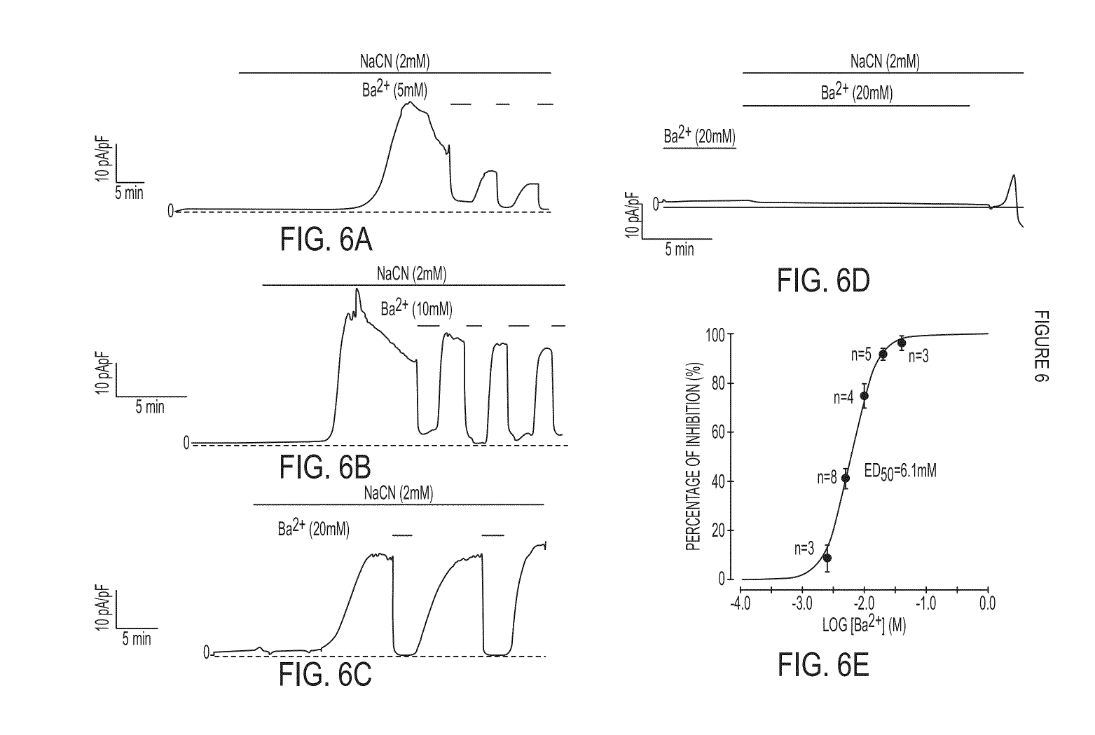

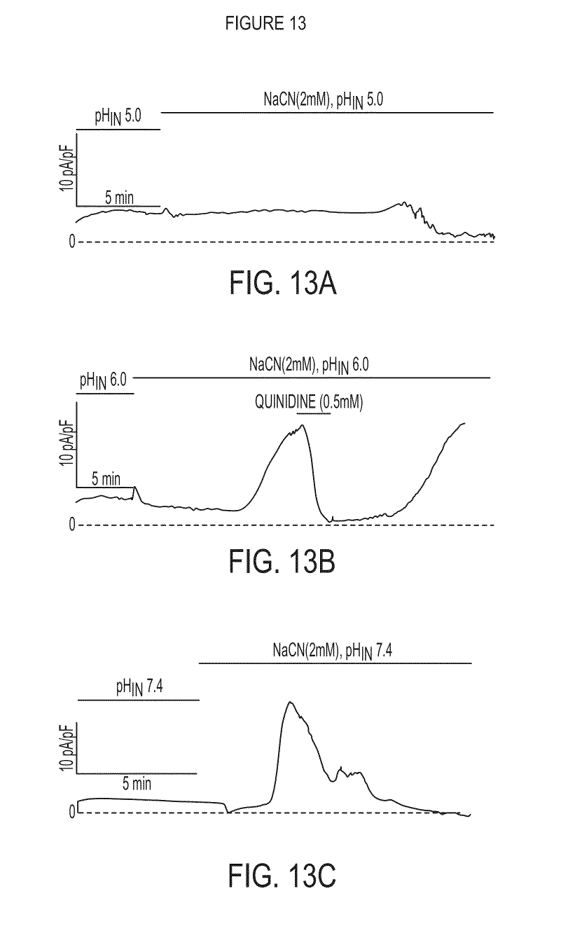

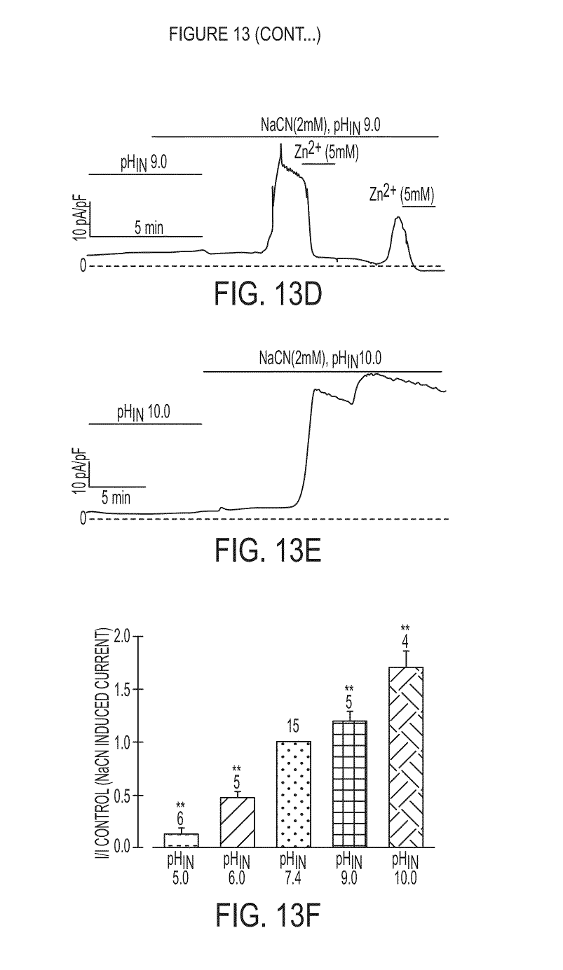

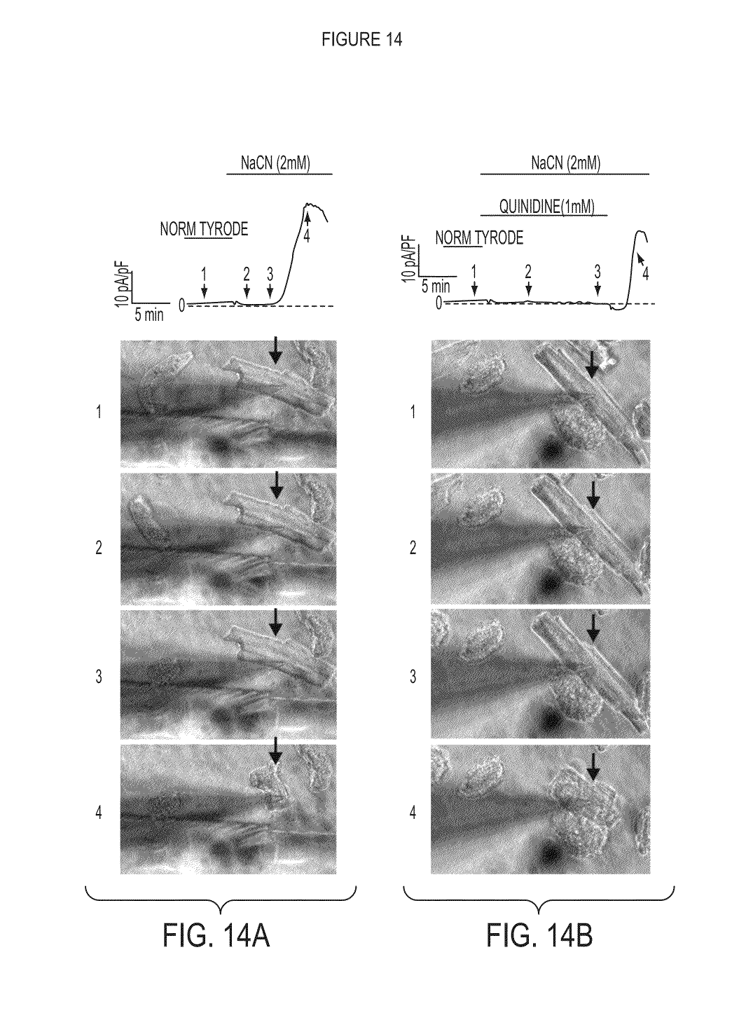

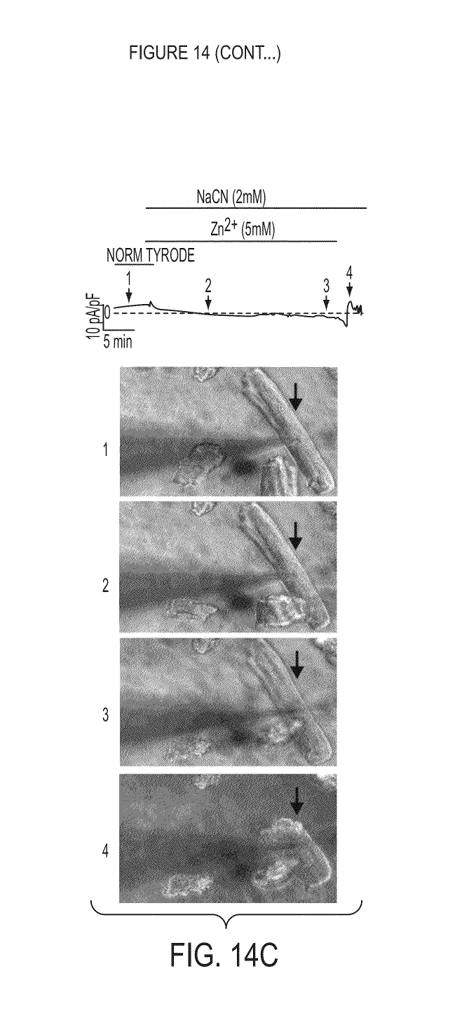

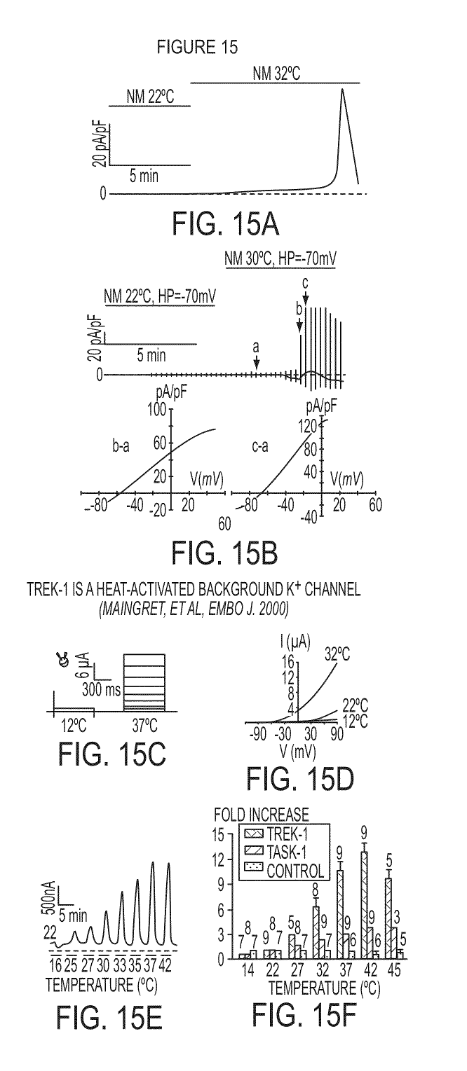

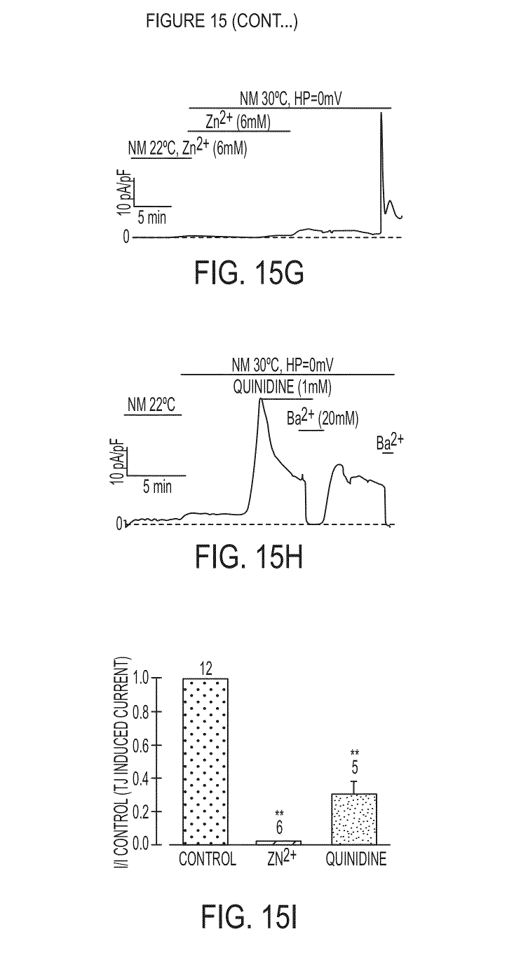

The NaCN induced current is decreased by reduction of intracellular pH. Sample traces of NaCN induced current in the presence of different intracellular pHs are shown in FIG. 13A-E. In the presence of internal pH of 7.4 (Control condition), a large outward current was induced by NaCN (FIG. 13C). Smaller outward currents were induced by NaCN at internal pHs of 5.0 (FIG. 13A) and 6.0 (FIG. 13B), however the peak current is significantly larger when the internal pH was increased to 9.0 (FIG. 8D and 10.0 (FIG. 13E), respectively. Moreover, the NaCN induced current was still sensitive to Zn²⁺ (FIG. 13D) and quinidine (FIG. 13B). The averaged data was normalized and compared in FIG. 13F. It clearly demonstrates that the NaCN induced current is significantly increased with increasing internal pH.

Preconditioning observed in the metabolic model of ischemia has mimicked that observed in the whole heart but controversy remains as to the identity of the current induced by NaCN. This current was assumed to be associated with surface K_{ATP} channels, since glibenclamide was shown to reduce the peak current (8; 26). However, further studies demonstrated the absence of inhibition of this current by glibenclamide in IPC (Liu et al., 1997 Am J Physiol. 273:H1637-43; Findlay 1993, Cardiovasc. Drugs Ther. 7 Suppl 3:495-497; Findlay, 1993 J. Pharmacol Exp Ther 266:456-467). Because of this uncertainty, more recently interest has focused on K_{ATP} channels of mitochondrial origin (Liu et al., 1998 Circulation 97:2463-2469, Liu et al., 1999 PIVAS 874:27-37) and their functional role in IPC was also considered (Dahlem et al. 2004, Biochim Biophys Acta 1656:46-56; Gross et al., 2003 Am J Physiol Heart Circ Physiol 285:H921-H930). The data presented herein indicates that this current was not carried by K_{ATP} channels from the surface membrane and that block of these channels in the mitochondrial membrane does not alter the amplitude of the NaCN induced current. The explanation for previous sets of results is that the NaCN induced current decays on its own and application of glibenclamide at the current's peak does not allow independent evaluation of the drug's action (Irie et al., 2003 Circulation 108 Suppl 1:II341-II347). Although the background K⁺ current IK₁ has been proposed to be involved in the IPC (Diaz et al., 2004 Cir Res 95:325-332), it does not exhibit the current-voltage relationship observed for the NaCN induced current. Moreover, this current is insensitive to extracellular Cs⁺ (a blocker of IK₁) and its relative insensitivity to barium (K_{d}=6.1 mM) also argues against this channel type. It has also been demonstrated that the NaCN induced current is insensitive to the classic K⁺ channel blockers (4AP). Further pharmacological evaluation with Zn²⁺ and quinidine indicate that the channel should belong to the family of non-traditional K⁺ channels, also known as K2P channels.

The K2P channel family members are distributed widely in human tissues, are particularly abundant in the brain, but are also present in the heart (Patel and Lazdunski, 2004 Pflugers Arch 448:261-273; Lesage and Lazdunski, 2000 Am J Physiol Renal Physiol 279:F793-F801). They consist of 16 members subdivided into five subfamilies named TWIK, TREK, TALK, THIK and TASK. All members are blocked by Zn²⁺ and quinidine but have different I/V relationships and dependence on the internal and external pH (Girard et al., 2004 Med Sci 20:544-549; Lesage 2003, Neuropharmacology 44:1-7). The finding that elevated external pH prevents the appearance of the outward current induced by NaCN are consistent with the experiments that methanandamide (specific blocker of TASK1-3) fails to abolish the current, suggesting that the TASK family (blocked by external acidosis and stimulated by external alkalosis), the first subfamily of K2P channel which was identified and extensively studied in the heart, is not the molecular correlate of the NaCN induced current (Barbuti et al. 2002 Am J Physiol Heart Circ Physiol 282:H2024-H2030). The NaCN induced current is increased at higher internal pH and decreased at higher external pH, sharing this pH dependence with the TWIK family (TWIK1-2) and TRAAK family of K2P channels (Lesage and Lazdunski 2000, Am J Physiol Renal Physiol 279:F793-F801). Recently, Liu, et al. reported the expression of K2P channel genes in adult rat heart with predominant expression of TWIK2, TASK-1 and TREK-1 in the ventricles (Liu and Saint, 2004 Clin Exp Pharmacol Physiol 31:174-178).

The present invention is not to be limited in scope by the specific embodiments described herein which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the claims. Throughout this application various publications are referenced. The disclosures of these publications in the entireties are hereby incorporated by reference into this application in order to more fully describe the state of the art to those skilled therein as of the date of the invention described and claimed herein.

What is claimed is:

1. A method, comprising
(i) contacting a K2P channel protein with a test compound under conditions and for a time sufficient to allow the K2P channel protein and the test compound to interact and bind, thus forming a complex,
(ii) detecting the complex, and
(iii) if a complex is formed, then identifying the test compound as a compound that binds to the K2P channel protein.

2. A method comprising,
(i) contacting a cell expressing a K2P channel protein with a test compound and determining the level of K2P channel protein activity;
(ii) in a separate experiment, contacting a cell expressing a K2P channel protein with a vehicle control and determining the level of K2P channel protein activity where the conditions are essentially the same as in (i),
(iii) comparing the level of K2P channel protein activity determined in (i) with the level determined in (ii), and
(iv) if the level of K2P channel protein activity in the presence of the test compound in (i) is increased compared to the level of K2P channel protein activity in (ii), then determining that the test compound is a K2P channel activator.

3. A method comprising,
(i) contacting a cell expressing a K2P channel protein with a test compound and a known K2P channel activator and determining the level of K2P channel protein activity;
(ii) in a separate experiment, contacting a cell expressing a K2P channel protein with a known K2P channel activator and a vehicle control, where the conditions are essentially the same as in (i),
(iii) comparing the level of K2P channel protein activity determined in (i) with the level determined in (ii), and
(iv) if the level of K2P channel protein activity in the presence of the test compound in (i) is decreased compared to the level of K2P channel protein activity in (ii), then determining that the test compound is a K2P channel protein inhibitor.

4. The method of claim 3, wherein the known channel activator is sodium cyanide (NaCN).

5. The method of claim 2 or claim 3, wherein the activity of the K2P channel protein is determined using patch clamp studies to measure the induced current.

6. The method of claim 2 or claim 3, wherein the activity of the K2P channel protein is determined by measuring RB efflux.

7. The method of claim 2 or claim 3, wherein the activity of the K2P channel protein is determined by measuring shrinkage of the cell expressing the K2P channel protein.

8. The method of claim 2 or claim 3, wherein the K2P channel protein is a recombinant K2P channel proteins or a K2P channel fusion protein.
